SEQUENCE LISTING (2 sequences)

SEQ ID NO: 1 - DNA, 19 bp, Artificial Sequence
  Synthetic oligonucleotide primer
ggattacaggyrtgagcca

SEQ ID NO: 2 - DNA, 18 bp, Artificial Sequence
  Synthetic oligonucleotide primer
rccaytgcactccagcct

(19) United States
(12) Patent Application Publication
(10) Pub. No.: US 2004/0081982 A1
(43) Pub. Date: Apr. 29, 2004

(54) Neocentromere-based mini-chromosomes or artificial chromosomes

(76) Inventors: Kong-Hong Andy Choo, Doncaster East (AU); Lee Hwa Wong, Vermont (AU); Richard Eric Saffrey, Glen Iris (AU)

Correspondence Address: BAKER & BOTTS, 30 ROCKEFELLER PLAZA, NEW YORK, NY, 10112

(21) Appl. No.: 10/463,981

(22) Filed: Jun. 17, 2003

Related U.S. Application Data
(63) Continuation of application No. PCT/AU01/01644, filed on Dec. 20, 2001.

(30) Foreign Application Priority Data
Dec. 21, 2000 (AU) PR2247/00
Nov. 16, 2001 (AU) PR8909/01

(51) Int. Cl. C12Q 1/68; C07H 21/04

(52) U.S. Cl. 435/6; 536/23.1

(57) ABSTRACT

The present invention is directed generally to a defined or isolated nucleic acid molecule encompassing a neocentreomere or a functional derivative thereof or a latent, synthetic or hybrid form thereof and its use inter alia in developing a range of eukaryotic mini-chromosomes and artificial chromosomes including mammalian (e.g. human) and non-mammalian mini-chromosomes and artificial chromosomes. The present invention further provides a telomere-associated chromosome truncation (TACT) approach to develop mini-chromosomes, but is not limited to this approach. It is directed to any truncation approach that produces a neocentromere-containing mini-chromosome, or any transfection approach using cloned or pre-fabricated DNA that provides neocentromere function in the construction of artificial chromosome. Such mini-chromosomes and artificial chromosomes are useful in a range of genetic therapies.

### CROSS-REFERENCE TO RELATED APPLICATIONS

This application is a continuation application of International Patent Application Ser. No. PCT/AU01/01644, filed Dec. 20, 2001, which was published in English as WO02/50288, and claims priority to Australian Provisional Patent Application Ser. No. PR2247/00, filed Dec. 21, 2000 and Australian Provisional Patent Application Ser. No. PR8909/01, filed Nov. 16, 2001, the contents of which are incorporated herein by reference in their entireties.

### FIELD OF THE INVENTION

The present invention is directed generally to a defined or isolated nucleic acid molecule encompassing a neocentreomere or a functional derivative thereof or a latent, synthetic or hybrid form thereof and its use inter alia in developing a range of eukaryotic mini-chromosomes and artificial chromosomes including mammalian (e.g. human) and non-mammalian mini-chromosomes and artificial chromosomes. The present invention further provides a telomere-associated chromosome truncation (TACT) approach to develop mini-chromosomes, but is not limited to this approach. It is directed to any truncation approach that produces a neocentromere-containing mini-chromosome, or any transfection approach using cloned or pre-fabricated DNA that provides neocentromere function in the construction of artificial chromosome Such mini-chromosomes and artificial chromosomes are useful in a range of genetic therapies.

### BACKGROUND OF THE INVENTION

Bibliographic details of the publications numerically referred to in this specification are collected at the end of the description.

Reference to any prior art in this specification is not, and should not be taken as, an acknowledgment or any form of suggestion that this prior art forms part of the common general knowledge in Australia or any other country.

The rapidly increasing sophistication of recombinant DNA technology is greatly facilitating research and development in the medical and allied health fields. A particularly important area is in mammalian, including human, genetics and the elucidation of the molecular mechanisms behind genetic abnormalities. Progress in research in this area has been hampered by the lack of a fully sequenced nucleic acid molecule containing a human centromere. The identification and/or cloning of such a molecule would promote the development of techniques for introducing genes into eukaryotic cells and in particular mammalian, including human, cells and will be an important asset to gene therapy and the development of expression systems for the production of a range of recombinant gene products in mammalian cells. Importantly, the identification and/or cloning of a fully sequenced centromeres facilitates the development of mammalian mini-chromosomes and artificial chromosomes.

Mammalian mini-chromosomes and artificial chromosomes have a variety of potential biotechnological and therapeutic applications arising from their ability to exist episomally and allow expression of genes under their endogenous control elements independently of the host genomic DNA. Because they are in effect fully functional mammalian chromosomes, there is no theoretically upper limit to the size of DNA that can be introduced into these entities. By analogy with their yeast counterparts, it has been assumed that mammalian mini-chromosomes and artificial chromosomes require a functional mammalian centromere, telomeres and DNA replication origins in order for proper segregation. At present, the least understood and most complex of these three components is the centromere.

The identification of an increasing number of protein components necessary for correct centromere activity, and the characterization of centromere DNA sequences in a variety of species, have greatly increased the knowledge of the mechanisms underlying centromere formation and function (1,2,3). This knowledge has facilitated the development of a number of strategies for mammalian mini-chromosome and artificial chromosome construction. One strategy involves the de novo formation of human artificial chromosomes by co-transfection of telomeric DNA with large arrays of human α-satellite into human cells (4,5,6,7). Studies using this strategy have shown that only α-satellite DNA-containing CENP-B boxes can participate in de novo artificial chromosome formation (5,8). While some of the generated artificial chromosomes were linear in structure (4), others were consistently circular (5,7,8). The artificial chromosomes ranged in size from ˜1-13 Mb and were typically one or more orders of magnitude larger than the input DNA. This increase in size has been attributed to end-joining of input DNA following transfection (4).

A different strategy involves the use of telomere-associated chromosome truncation to remove non-essential chromosomal materials around a normal centromere to produce a mini-chromosome in situ. Sequential truncation of a human X chromosome has yielded a 2.5-Mb mini-chromosome comprising approximately 1.8 Mb of X-chromosome α-satellite DNA and 400 kb of proximal Xp DNA (9,10,11). This chromosome shows mitotic stability comparable to that of the normal human X chromosome (10,11). A similar approach has produced a number of human Y chromosome-derived mini-chromosomes, ranging in size from ˜0.7 Mb to over 4 Mb, with the smaller ones being relatively unstable (12,13). The larger mini-chromosomes were stably maintained in CHO cells, chicken DT40 cells and mouse L cells but showed poor stability when introduced into mouse ES cells, suggesting differential requirements for correct centromere function in different cell types (14,15).

A third strategy for production of mammalian artificial chromosomes involves the amplification of pericentric DNA followed by controlled breakage of chromosomes to produce satellite DNA-based artificial chromosomes of between 60 and 400 Mb (16,17,18,19,20).

In recent years, neocentromeres (NCs) that lack the repeat sequences traditionally associated with centromere function have been described in humans (21) and Drosophila (22). Characterization of the underlying DNA and protein-binding profile of neocentromeres in humans has suggested an epigenetic mechanism of centromere formation independent of primary DNA sequence composition (23,24,25,26). The discovery of neocentromeres provides an alternative approach to the construction of mini-chromosomes and artificial chromosomes to those previously described that have been based on the use of repeated centromere DNA sequences.

In accordance with the present invention, the inventors have generated a series of mitotically stable, human mini-chromosomes containing a fully functional human neocentromere. The generation of a human mini-chromosome permits the development of genetic therapies, transgenic plant and animal production and recombinant protein production.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier number (SEQ ID NO:). The SEQ ID NOs: correspond numerically to the sequence identifiers <400>1, <400>2, etc. A sequence listing is provided after the claims.

The present invention is predicated in part on the use of a telomere-associated chromosome truncation (TACT) approach to develop mini-chromosomes. Truncation constructs are developed comprising a selectable marker, a targeting DNA sequence homologous to a small region on the p′ or q′ arms of a eukaryotic chromosome such as human mardel(10), and a small array of telomeric sequences. p′ and q′ arms refer to the short and long arms of the mardel(10) chromosome but also refer generally to the short and long arms of any marker chromosome containing a neocentromere. A first truncation construct is transfected into a target cell and the marker selected. This results in a target chromosome with a truncated p′ or q′ arm depending on the truncation construct used. A second truncation construct is then transfected into the same cell employing the other of the p′ or q′ arm truncation construct. Again, following selection, a truncated form of the p′ or q′ arm of the target chromosome is obtained. The resulting mini-chromosome may then be isolated and used for gene therapy or gene expression. A modified targeting method that may or may not include a telomere DNA may also be used to introduce genes or other nucleotide sequences into a target chromosome.

One aspect of the present invention provides an isolated nucleic acid molecule comprising a sequence of nucleotides which defines an eukaryotic neocentromere.

More particularly, the present invention provides an isolated nucleic acid molecule comprising a sequence of nucleotides derived from a eukaryotic chromosome and encompassing a neocentromere, which nucleic acid molecule exists in a truncated or modified form in a compatible cell or, when introduced by transfection as a pre-fabricated DNA entity into a compatible cell, is capable of replicating, acting as an extra chromosomal element and segregating with cell division.

Another aspect of the present invention contemplates the use of a method for identifying a neocentromere or a functional homolog, said method comprising isolating DNA by chromatin immunoprecipitation using an antibody specific to mammalian CENP-A and/or CENP-C or an antibody capable of cross interacting with mammalian CENP-A and/or CENP-C, amplifying the DNA isolated by immunoprecipitation and incorporating label into the amplified DNA and then using the amplified DNA to probe a DNA array comprising genomic DNA or its equivalent and identifying and isolating clones which hybridize to said immunoprecipitated DNA.

A further aspect of the present invention provides an isolated nucleic acid molecule in the form of a human neocentromere-based mini-chromosome (NC-MiCs) comprising a neocentromere or a latent, synthetic or hybrid form thereof which enable stable segregation during cell division.

Yet another aspect of the present invention provides a method for generating a mini-chromosome, said method comprising:

introducing into a human or mammalian cell which carries a chromosome containing a neocentromere, a truncation construct comprising a vector having telomeric sequences, a selectable marker, homologous targeting DNA on one or other of the q′ or p′ arm of the target chromosome;

selecting for cells expressing the selectable marker;

introducing into said cells a second truncation construct comprising the other of said q′ or p′ targeting DNA;

selecting for cells expressing the selectable marker associated with said second truncation construct; and

then isolating the truncated chromosome which comprises a neocentromere.

### BRIEF DESCRIPTION OF THE FIGURES

FIG. 1 is a diagrammatic representation showing neocentromere-based mini-chromosomes (abbreviated as NC-MiC) formation from the mardel(10) chromosome. (A) Structure of TACT targeting constructs. Targeting DNA from the p′ or q′ arms of mardel(10) were cloned into vectors containing small arrays of cloned human telomeric DNA (Htel) adjacent to a mammalian selectable marker (hygromycin^{R}, neomycin^{R}, or puromycin^{R}). Constructs were linearized at a restriction site between the vector DNA and the telomere repeats to expose the telomere sequences at the terminal. Following a homologous recombination event, the vector DNA should not be incorporated into the host chromosome. Loss of vector DNA allowed design of a screening assay for possible recombination events. (B) Schematic formation of mardel(10) and NC-MiCs. Open arrows indicate the breakpoints on the normal chromosome 10 in the generation of mardel(10). The long and short arms of mardel(10) are denoted as q′ and p′, respectively. The truncation events resulting in the formation of NC-MiCs 1-5 are represented by Tc1 and Tc2. Location of the zeocin resistance gene inserted into mardel(10) is indicated by an asterisk. Position of BAC B10K1 used in a screening assay for loss of p′ arm is shown. Bold lines above chromosome 10 denote the locations and designations of subchromosomal DNA paints used to characterize the NC-MiCs. (C) Mapping of the NC-MiCs. Ordered cosmid and BAC clones covering approximately 3 Mb and previously mapped to the 10q25 neocentromere region are shown. Vertical shaded area represents the E8 BAC containing the centromere protein-binding neocentromere (NC) domain (23,27). Open arrowheads to the left and right of E8 indicate intended positions of targeted truncation using q′ and p′ truncation constructs. (+) denotes a positive FISH result for a BAC or cosmid probe on an NC-MiC, while ⁻( indicates a negative FISH result. The approximate sizes of the different NC-MiCs are shown in parentheses. (D) Characterization of the targeted q′ truncation site. The targeting DNA for q′ truncation is a 6-kb XbaI fragment subcloned from cosmid Y3C94 (also present in BAC B79E16) and is represented by the hatched box. Locations of PCR probes from B79E16 are indicated by double-headed arrows and are denoted F1, F2, and F3. Hybridization status of these probes on NC-MiCs is denoted by (+) or (−)confirming that the q′ truncation was the result of a targeted event.

FIG. 2 is a photographic representation of FISH and/or immunofluorescence analysis of ZB30 and NC-MiC2. (A) FISH analysis of ZB30 showing hybridization of E8 (green) and tagging of mardel(10) (arrowhead) with zeocin resistance gene (red). (B-H) FISH and/or immunofluorescence analysis on NC-MiC2. Normal chromosome 10 is indicated by arrowhead and NC-MiC2 by arrow. (i-iv) Combined image, and split images for green, red, and DAPI, respectively. (B) FISH using E8 (green) and Y3C94 cosmid probe (red), showing presence of Y3C94 on NC-MiC2. (C) FISH using E8 (green) and q′ cosmid Y3C109 (red), showing absence of Y3C109 on NC-MiC2. (D) FISH using E8 (green) and a PCR fragment F1 (red) derived from Y3C94/B79E16 and overlapping the q′ targeting DNA (see FIG. 1D), showing presence of F1 DNA on NC-MiC2. (E) FISH using E8 (green) and a PCR fragment F2 (red) derived from B79E16 in a region immediately distal to the targeting DNA (see FIG. 1D), showing absence of F2 DNA on NC-MiC2. (F) FISH using E8 (green) and immunofluorescence using anti-CENP-B antibody (red), showing absence of CENP-B on NC-MiC2. (G) FISH using E8 (green) and immunofluorescence using anti-CENP-E antibody (red), showing presence of CENP-E protein on NC-MiC2. (H) FISH using E8 (green) and telomere-repeat PNA probe (red), showing telomeric sequences on NC-MiC2.

FIG. 3 is a photographic representation of FISH and/or immunofluorescence analysis of NC-MiC3. Normal chromosome 10 is indicated by arrowhead and NC-MiC3 by arrow. (i-iv) Combined image, and split images for green, red, and DAPI, respectively. (A) FISH using E8 (green) and q′ BAC B79E16 (red), showing presence of B79E16 on NC-MiC3. (B) FISH using E8 (green) and q′ cosmid Y3C109 (red), showing absence of Y3C109 on NC-MiC3. (C) FISH using E8 (green) and p′ cosmid Y13C12 (red), showing presence of Y13C12 on NC-MiC3. (D) FISH using E8 (green) and p′ BAC B179N3 (red), showing absence of B179N3 on NC-MiC3. (E) FISH using E8 (green) and a pan-α-satellite pTRA-7 probe (red), showing absence of α-satellite on NC-MiC3. (F) FISH using E8 (green) and immunofluorescence using anti-CENP-E antibody (red), showing presence of CENP-E on NC-MiC3. (G) FISH using E8 (green) and immunofluorescence using anti-CENP-F antibody (red), showing presence of CENP-F on NC-MiC3.

FIG. 4 is a photographic representation of FISH and/or immunofluorescence analysis of NC-MiC4. Normal chromosome 10 is indicated by arrowhead and NC-MiC4 by arrow. (i-iv) Combined image, and split images for green, red, and DAPI, respectively. (A) FISH using E8 (green) and p′ cosmid Y13C12 (red), showing the presence of Y13C12 on NC-MiC4. (B) FISH using E8 (green) and p′ BAC B43A11 (red), showing the absence of B43A11 on NC-MiC4. (C) FISH using E8 (green) and pan-α-satellite pTRA-7 probe (red), showing absence of α-satellite on NC-MiC4. (D) FISH using TTAGGG telomere-repeat PNA probe, showing positive signals on all human telomeres but not on NC-MiC4. (E) FISH using E8 (green) and immunofluorescence using anti-CENP-B antibody (red), showing absence of CENP-B on NC-MiC4. (F) FISH using E8 (green) and immunofluorescence using anti-CENP-C antibody (red), showing presence of CENP-C on NC-MiC4. (G) FISH using E8 (green) and immunofluorescence using anti-CENP-E antibody (red), showing presence of CENP-E on NC-MiC4. (H) FISH using E8 (green) and immunofluorescence using anti-CENP-F antibody (red), showing presence of CENP-F on NC-MiC4.

FIG. 5 is a photographic representation of FISH and/or immunofluorescence analysis of NC-MiC5. Normal chromosome 10 is indicated by arrowhead and NC-MiC5 by arrow. (i-iv) Combined image, and split images for green, red, and DAPI, respectively. (A) FISH using E8 (green) and p′ BAC BA48L24 (red), showing presence of BA48L24 on NC-MiC5. (B) FISH using E8 (green) and p′ BAC BA69K10 (red), showing absence of BA69K10 on NC-MiC5. (C) FISH using E8 (green) and pan-α-satellite pTRA-7 probe (red), showing absence of α-satellite on NC-MiC5. (D) FISH using E8 (green) and immunofluorescence using anti-CENP-B antibody (red), showing absence of CENP-B on NC-MiC5. (E) FISH using E8 (green) and immunofluorescence using anti-CENP-A antibody (red), showing presence of CENP-A on NC-MiC5. (F) FISH using E8 (green) and immunofluorescence using anti-CENP-C antibody (red), showing presence of CENP-C on NC-MiC5. (G) FISH using E8 (green) and immunofluorescence using anti-CENP-E antibody (red), showing presence of CENP-E on NC-MiC5.

FIG. 6 is a photographic representation of chromosome-painting analysis of NC-MiCs. (A) Whole-chromosome paints for all 24 human chromosomes (1-22, X, Y). Left panel shows positive painting (green) on normal human chromosomes, except for the pericentric heterochromatic regions and those on the short arms of acrocentric chromosomes, and the q12 region of Y. Upper right panel shows the corresponding painting result for NC-MiC4 while lower right panel shows the result for NC-MiC5. All chromosome paints with the exception of chromosome 10 are negative on the NC-MiCs as evidenced by a lack of overlapping paint (green) and E8 (red) signals (confirmed by splitting individual images; not shown), while the chromosome-10 paint signal is clearly seen on NC-MiCs as evidenced by an overlap of red and green signals producing a combined yellow colour. (B) Subchromosome-10 paints on NC-MiC5. Positions for these subchromosome paints on chromosome 10 are shown schematically in FIG. 1B. Left-hand panels show designations of the paints and FISH results (green) on normal chromosome 10. Right-hand panels show the negative results obtained for these paints on NC-MiC5. E8 signals are in red. Chromosomes and NC-MiCs are not shown to scale.

FIGS. 7A-C are diagrammatic representations showing NC-MiC6 construction via the truncation of NC-MiC2. (A) Structure of TACT targeting constructs used for truncating the p′ arm from NC-MiC2. Targeting DNA from the p′ arm of mardel(10) and a mammalian selectable marker (blasticidin resistance gene, blasticidin^{R}) were cloned into vectors containing small arrays of cloned human telomeric DNA (Htel) adjacent to an antibiotic resistance gene, neomycin^{R}. Constructs were linearized at a restriction site between the vector DNA and the telomere repeats to expose the telomere sequences at the terminal. Following a homologous recombination event, the blasticidin resistance gene should not be incorporated into the host chromosome. Loss of the blasticidin resistance gene allowed the design of a screening assay for possible recombination events. (B) Schematic formation of mardel(10) and NC-MiCs. Open arrows indicate the breakpoints on the normal chromosome 10 in the generation of mardel(10). The long and short arms of mardel(10) are denoted as q′ and p′, respectively. NC-MiC2 was truncated at B79e16/Y3C94 (Tc1) (as described earlier) from mardel(10), whereas NC-MiC6 is a result of truncation 2, Tc2, at B137iL/Y13c15 using the TACT construct described in (A). (C) Mapping of the NC-MiCs 2 and 6. Ordered cosmid and BAC clones covering approximately 3 Mb and previously mapped to the 10q25 neocentromere region are shown. the vertical shaded area represents the centromere protein CENP-A-binding domain (51). Open arrowheads indicate positions of targeted truncation. (+) denotes a positive FISH result for a BAC or cosmid probe on an NC-MiC, while (−) indicates a negative FISH result. The approximate sizes of the different NC-MiCs are shown in parentheses.

FIG. 8 is a tabular and graphical representation showing the results of culturing NC-MiC6 for 60 divisions either in the presence or absence of neomycin drug G418 at a concentration of 150 μg/ml before they were harvested at various intervals for determination of stability. The X axis represents the number of divisions, whereas the Y axis represents the percentage of NC-MiC present in culture as determined by FISH analysis. The results indicated mitotic stability of NC-MiC-6 in the presence or absence of G418.

### DETAILED DESCRIPTION OF THE PREFERRED EMBODIMENTS

The present invention is predicated in part on the identification and isolation of nucleic acid molecules exhibiting neocentromeric properties. In accordance with the present invention, a neocentromere is considered a centromere which does not contain substantial amounts of repetitive DNA sequences that are present on a normal centromere (e.g. α-satellite in humans and minor satellite in mouse) and, when activated, is capable of functioning as a centromere. For example, a mammalian (e.g. human) neocentromere is a centromere which does not contain substantial α-satellite DNA repeat sequences. The term “substantial” in this context means that the nucleic acid molecule does not contain detectable normal centromeric repetitive DNA sequences such as α-satellite by FISH analysis under medium stringency conditions or by direct sequence comparison under medium homology criteria. The neocentromere may, however, contain a small number of highly diverged normal centromeric repetitive DNA sequences. In primates, for example, α-satellite DNA is considered to be about 170 bp in length. A nucleic acid molecule containing an activated neocentromere or a neocentromere otherwise functioning as a centromere facilitates in accordance with the present invention, the nucleic acid molecule in the form of a mini-chromosome or pre-fabricated with other DNA to facilitate transfection, replicating, remaining extra-chromosomal and segregating with cell division. Reference herein to “neocentromere” is taken to mean a centromere substantially devoid of repetitive DNA sequences that are normally present on the centromere of an endogenous and structurally unaltered chromosome such as α-satellite DNA repeat sequences on human chromosomes. Furthermore, a neocentromere is considered to be derived from a normally non-centromeric region of the genome.

Accordingly, one aspect of the present invention provides an isolated nucleic acid molecule comprising a sequence of nucleotides which defines a eukaryotic neocentromere.

More particularly, the present invention provides an isolated nucleic acid molecule comprising a sequence of nucleotides derived from a eukaryotic chromosome and encompassing a neocentromere, which nucleic acid molecule exists in a truncated or modified form in a compatible cell or, when introduced by transfection as a pre-fabricated DNA entity into a compatible cell, is capable of replicating, acting as an extra chromosomal element and segregating with cell division.

The present invention is exemplified herein by the identification of a human neocentromere. This is done, however, with the understanding that the present invention extends to all eukaryotic neocentromeres such as from mammalian, plant, avian, insect, worm, fungal, yeasts and reptilian chromosomes. The most preferred neocentromere, however, is from human chromosomes and their mammalian homologs.

The present invention is predicated in part on the construction of a number of neocentromere-based human mini-chromosomes using a combination of targeted telomere-associated truncation of the q′ arm and apparently random truncation of the p′ arm of the mardel(10) chromosome. The latter refers to a chromosome identified in a human patient and results from a re-arrangement of human chromosome 10. The mardel(10) marker is mitotically stable and, in accordance with the present invention, contains a functional neocentromere at a location regarded as non-centromeric. The neocentromere at mardel(10) is located between q24 and q26 on chromosome 10 and more particularly around q25. Even more particularly, the neocentromere maps to q25.2 on chromosome 10. The present invention is exemplified by DNA cloned from the q24-q26 region of the mardel(10) chromosome as well as the corresponding region on normal human chromosome 10. These DNA molecules contain a functional neocentromere. The present invention extends, however, to any neocentromere on any chromosome in mammalian and non-mammalian animals as well as plants, insects, worms, fungal, reptiles and yeasts.

Detailed mapping using cosmid and BAC probes from the 10q25 region allowed the truncation sites to be defined and the demonstration that the NC-MiCs contain single-copy intact DNA from this region. Extensive FISH using pan-α-satellite DNA, whole-chromosome paints for all human chromosomes and different subchromosomes-10 paints, revealed that none of the NC-MiCs have acquired detectable amounts of α-satellite DNA or other human genomic sequences. Based on the mapping data, the sizes of NC-MiCs 3, 4 and 5, were estimated to be approximately 1.6, 1.6 and 0.8 Mb, respectively.

The present invention further contemplates a nucleic acid molecule or its chemical equivalent having a tertiary structure which defines a human neocentromere or a functional derivative thereof or a latent, synthetic or hybrid form thereof or its mammalian or non-mammalian homolog.

Even more particularly, the present invention is directed to an isolated nucleic acid molecule having a sequence of nucleotides or their chemical equivalents which directs a conformation defining a human neocentromere or a functional derivative thereof or a latent, synthetic or hybrid form thereof or its mammalian or non-mammalian homolog wherein the centromere associates with centromere binding proteins such as CENP-A and CENP-C but not limited to these proteins.

Reference herein to “latent” in relation to a centromere includes reference to a centromere not normally functional but nevertheless activatable under certain conditions. A latent centromere may also be considered as a neocentromere provided it has no substantial repetitive DNA sequences which are found on normal centromeres such as α-satellite DNA repeat sequences. A preferred repetitive DNA sequence in a non-human chromosome is considered to be a functionally equivalent repeat DNA to α-satellite DNA.

Accordingly, another aspect of the present invention contemplates the use of any method for identifying a neocentromere or a functional homolog, said method includes isolating DNA by chromatin immunoprecipitation using an antibody specific to mammalian CENP-A and/or CENP-C, or an antibody capable of cross interacting with mammalian CENP-A and/or CENP-C, amplifying the DNA isolated by immunoprecipitation and incorporating label into the amplified DNA and then using the amplified DNA to probe a DNA array comprising genomic DNA or its equivalent and identifying and isolating clones which hybridize to said immunoprecipitated DNA (52).

The size of the neocentromere in accordance with the present invention may range from about 50 bp to about 2000 kbp, from about 70 bp to about 1000 kbp, from about 75 bp to about 800 kpb, from about 80 bp to about 500 kpb, from about 85 bp to about 200 kpb, from about 90 bp to about 100 kbp, from about 100 bp to about 1 kpb, about 120 bp to about 500 bp, about 180 bp to about 300 bp. In one embodiment, the neocentromere is approximately 60-100 kbp. In another embodiment, the neocentromere is about 80 kpb. In a particularly preferred embodiment, the neocentromere is from about 50 kbp to about 2000 kpb. The neocentromere may encompass different structurally or functionally distinct domains such as CENP-A-binding or other centromere protein-binding domain, or domains showing different replication timing, chromatin structure, scaffold organisation, chemical modification status (e.g. acetylation, methylation, phosphorylation, poly-ADP-ribosylation). Provision is also given to the size of the neocentromere being larger than 2000 kbp to encompass all the functionally important domains.

The size of the mini-chromosome in accordance with the present invention may range from about 500 bp to about 20000 kbp, from about 700 bp to about 1000 kbp, from about 750 bp to about 8000 kpb, from about 800 bp to about 5000 kpb, from about 850 bp to about 2000 kpb, from about 900 bp to about 1000 kbp, from about 1000 bp to about 10 kpb, about 1200 bp to about 5000 bp, about 1800 bp to about 3000 bp. In one embodiment, the mini-chromosome is approximately 600-1000 kbp. In another embodiment, the mini-chromosome is about 800 kpb. In a particularly preferred embodiment, the mini-chromosome is from about 500 kbp to about 20000 kpb. The mini-chromosome may encompass different structurally or functionally distinct domains such as a neocentromere, replication origins, and telomeres. In a particularly preferred embodiment, the mini-chromosome contains an active neocentromere.

The nucleic acid molecule of the present invention may comprise a naturally occurring nucleotide sequence from a healthy human subject or may comprise the nucleotide sequence from a human subject exhibiting one or more chromosomal-dependent conditions such as a subject carrying mardel(10) chromosome or a chromosome conferring an equivalent or similar condition or may carry one or more nucleotide substitutions, deletions and/or additions relative to the naturally or non-naturally occurring sequence. Such modifications are referred to herein as “derivatives” and include mutants, fragments, parts, homologs and analogs of the naturally occurring nucleotide sequence. Preferably, the derivatives of the present invention still define a functional neocentromere.

Reference herein to a “neocentromere” includes reference to a functional neocentromere or a functional derivative thereof meaning that it is capable of facilitating sister chromatid cohesion and chromosomal segregation during mitotic cell divisions and/or is capable of associating with CENP-A and/or CENP-C and/or other functionally important centromere proteins and/or is capable of interacting with anti-CENP-A antibodies or anti-CENP-C antibodies or antibodies to other functionally important centromere proteins. Generally, and preferably, the neocentromere is incapable of interacting with CENP-B or anti-CENP-B antibodies. Alternatively, the neocentromere may be a latent centromere capable of activation by epigenetic mechanisms or other relevant mechanisms. The neocentromere may also be a hybrid of other human, mammalian, plant or yeast neocentromeres. Synthetic neocentromeres provided by, for example, polymeric techniques to arrive at the correct conformation are also contemplated by the present invention. All such forms and definitions of neocentromeres are encompassed by use of this term.

Another aspect of the present invention provides an isolated nucleic acid molecule or chemical equivalent which comprises a nucleotide sequence or chemical equivalent directing a conformation which defines a neocentromere or a functional derivative thereof or a latent, synthetic or hybrid form thereof and wherein said neocentromere is substantially devoid of normal centromeric repetitive DNA such as α-satellite DNA and wherein the neocentromere is capable of associating with CENP-A or CENP-C or other functionally important centromere-binding proteins or antibodies thereto.

Preferably, the neocentromere is incapable of interacting with CENP-B or antibodies thereto.

In one embodiment, the neocentromere corresponds to a human genomic region which maps between q24 and q²⁶ on chromosome 10 and in particular q25 on chromosome 10.

The nucleic acid molecule or its chemical equivalent of the present invention defining a conformational neocentromere or functional derivative thereof or latent, synthetic or hybrid form thereof is useful inter alia for the generation of mini-chromosomes or artificial chromosomes such as human neocentromere-based mini-chromosomes (NC-MiCs), human artificial chromosomes (HACs), mammalian artificial chromosome (MACs), yeast artificial chromosomes (YACs) and plant artificial chromosomes (PLACs). Human NC-MiCs are particularly useful since they are capable of accommodating large amounts of DNA and are capable of propagation in human cells. The NC-MiCs are non-viral in origin and, hence, are more suitable for gene therapy by, for example, introducing therapeutic genes, than conventional viral based vector systems. Furthermore, the NC-MiCs remain extra-chromosomal and, hence, have no insertional/substitutional mutagenic potential. The essence of a NC-MiCs is the presence of a neocentromere or latent, synthetic or hybrid form thereof which enables stable segregation during cell division. The NC-MiCs also remain extra-chromosomal and, hence, are more suitable for gene therapy. Reference to “extra-chromosomal” means that it does not integrate into the main chromosome and, in effect, is episomal.

Accordingly, the present invention provides a genetic construct comprising an origin of replication for a eukaryotic cell and a nucleic acid molecule encompassing a eukaryotic neocentromere or a functional derivative thereof or a latent, synthetic, hybrid form thereof or its mammalian or non-mammalian homolog flanked by telomeric nucleotide sequences functional in the cell in which the genetic construct is to replicate and where said genetic construct when introduced into a cell is a replicating, extra chromosomal element, either in a circular or linear form, which segregates with cell division.

More particularly, the present invention further contemplates a genetic construct in the form of a mini-chromosome or an artificial chromosome comprising an origin of replication for a mammalian, human, plant or yeast cell and a nucleic acid molecule encompassing a human neocentromere or a functional derivative thereof or a latent, synthetic or hybrid form thereof or its mammalian or non-mammalian homolog flanked by telomeric nucleotide sequences or in a circular form carrying minimal or no telomere sequences functional in the cell in which the mini-chromosome or artificial chromosome is to replicate.

Another embodiment provides a genetic construct in the form of a mini-chromosome or an artificial chromosome comprising an origin of replication for a mammalian, human, plant or yeast cell and a nucleic acid molecule having a tertiary structure which defines a human neocentromere or a functional derivative thereof or a latent, synthetic or hybrid form thereof or its mammalian homolog flanked by telomeric sequences or in a circular form carrying minimal or no telomere sequences functional in the cell in which the mini-chromosome or artificial chromosome is to replicate.

Yet another embodiment is directed to a genetic construct in the form of a mini-chromosome or an artificial chromosome comprising an origin of replication for a mammalian, human, plant or yeast cell and a nucleic acid molecule having a sequence of nucleotides which directs a conformation defining a human neocentromere where the centromere associates with CENP-A and/or CENP-C and/or other centromere proteins or antibodies thereto and does not contain substantial α-satellite DNA repeat sequences, said nucleic acid molecule flanked by telomeric nucleotide sequences or in a circular form carrying minimal or no telomere sequences functional in the cell which the mini-chromosome or artificial chromosome is to replicate.

The present invention further provides a method for generating a mini-chromosome, said method comprising:

introducing into a cell a truncation construct comprising a vector having telomeric sequences, a selectable marker, homologous targeting DNA on one or other of a q′ or p′ arm of the target chromosome;

selecting for cells expressing the selectable marker;

introducing into said cells a second truncation construct comprising the other of said q′ or p′ targeting DNA;

selecting for cells expressing the selectable marker associated with said second truncation construct; and

then isolating the truncated chromosome which comprises a neocentromere.

In one embodiment, the q′ and p′ targeting arms target the mardel(10) chromosome.

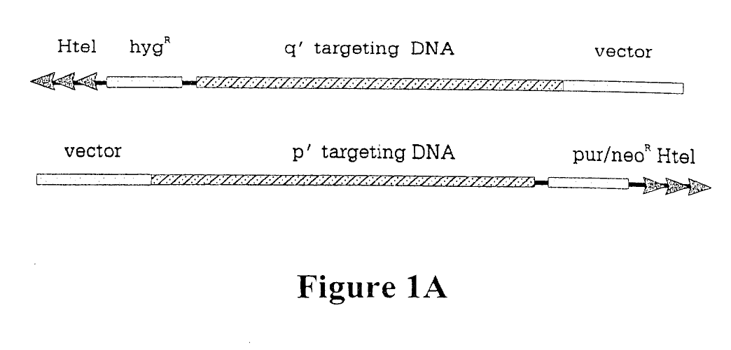

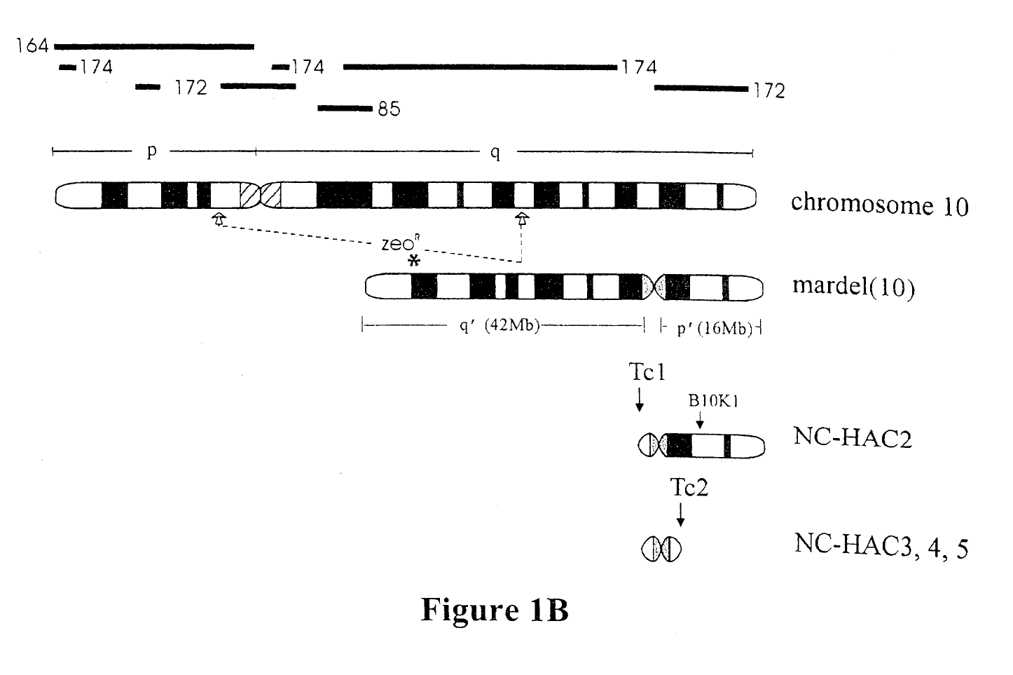

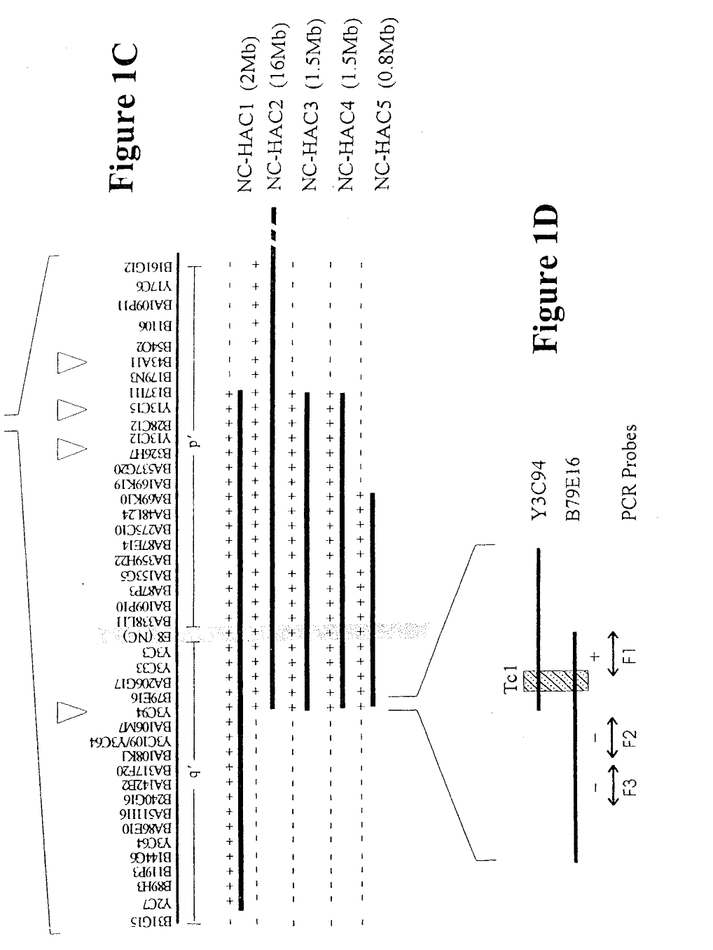

In particularly preferred embodiments, the mini-chromosomes are NC-MiCs 1-5 as described in FIG. 1.

The present invention further provides a cell comprising a chromosome having q′ and p′ arms flanking a neocentromere wherein one or both q′ and p′ arms are truncated.

The above described method or its modified version thereof may also be used to introduce a gene or other nucleotide sequence such as for expression.

The present invention extends to eukaryotic cells such as human, primate, insect, yeast or other eukaryotic cells carrying the genetic constructs of the present invention and to proteins produced therefrom.

The genetic constructs of the present invention include mini-chromosomes and artificial chromosomes as well as DNA constructs useful in the generation of mini-chromosomes and artificial chromosomes.

The genetic constructs may also comprise marker genes, unique restriction sites, or recombination enhancing markers (e.g. LoxP DNA, for use with Cre recombinase) to facilitate insertion of adventitious DNA. Accordingly, the genetic constructs of the present invention may further comprise adventitious or heterologous DNA encoding a product of interest. Preferred products of interest include biotechnologically or pharmaceutically useful genes, such as genes encoding cytokines, receptors, growth regulators and the like. Endogenous genes may also be replaced by wild-type genes or modified genes.

Specific DNA sequences intrinsically present on the genetic constructs (e.g. putative ESTs or expressed genes or unstable DNA elements) may also be identified, removed or modified by further genetic manipulation or engineering. Other DNA components (e.g. therapeutic or marker genes, LoxP DNA sequences, unique restriction sites such as I-SceI) may also be subsequently added to the genetic constructs to improve or broaden their utility or application.

The adventitious or heterologous DNA may also encode a molecule not synthesized in a sufficient amount in a particular subject and hence the increased copy number permits greater amounts of the molecule being synthesized.

Accordingly, the present invention contemplates a genetic construct comprising an origin of replication and a first nucleic acid molecule defining a human neocentromere or a functional derivative thereof or latent, synthetic or hybrid form thereof or a mammalian or non-mammalian homolog, a second nucleic acid molecule encoding a peptide, polypeptide or protein, wherein said first and second nucleic acid molecules are flanked by human telomeric sequences or in a circular form carrying minimal or no telomeric sequences functional in the cell in which the genetic construct is to replicate.

Reference herein to “segregate” preferably means mitotically stable segregation. Conveniently, stable segregation may be determined as the presence of a mini-chromosome or an artificial chromosome in more than 40-60% of daughter cells after 4-6 months of continuous passage.

The present invention extends to other mini-chromosomes or artificial chromosome analogs to the NC-MiCs described above such as HACs, MACs and PLACs, or similar entities by any other descriptions or names such as micro-chromosomes, synthetic chromosomes, and variations thereof.

Another aspect of the present invention relates to peptides, polypeptides and proteins which bind, interact or otherwise associate with the human neocentromere of the present invention or its mammalian and non-mammalian homolog. Preferably, the molecules are proteins, referred to as primary (1°) proteins. The 1° proteins bind to the neocentromere and secondary (2°) proteins bind to the 1° proteins before or after association with the neocentromere. The identification of the human neocentromere in accordance with the present invention provides a mechanism for assaying 1° proteins and 2° proteins which may be important for screening chromosomes in, for example, genetic disorders. This is particularly the use in Down's Syndrome which results from defective chromosome segregation.

The 1° proteins are readily detected by, for example, a gel shift assay. The nucleic acid molecule of the present invention defining the human neocentromere is digested, labelled and contacted with nuclear extract putatively containing the 1° proteins and resolved on a gel. When a 1° protein binds to a fragment carrying a binding portion of the neocentromere, the DNA fragment migrates in the gel at a slower rate due to the bound protein.

The present invention extends to purified 1° proteins capable of association with the subject centromere and to genetic sequences encoding same and to antibodies thereto.

The neocentromeres of the present invention are readily identified and characterized using, for example, human fibrosarcoma cell lines. For example, DNA suspected of carrying a neocentromere is introduced into fibrosarcoma cells in a linear form, generally together with a telomeric sequence. The cells are then screened for the presence of replicating, extra chromosomal and segregating elements, referred to as artificial chromosomes.

The present invention further encompasses eukaryotic cells carrying replicating, extra-chromosomal and segregation nucleic acid molecules. Preferably, the eukaryotic cells are mammalian cells and most preferably human cells. The nucleic acid molecules according to this aspect of the present invention are preferably as herein described.

The following cell lines were deposited at the ECACC, Centre for Applied Microbiology and Research, Porton Down, Salisbury, Wilts, SP4 0JG, United Kingdom on 20 Dec. 2000, under the listed Accession Numbers:

| Cell Line | Acronyn | Accession Number |
|---|---|---|
| CHO/BE ZB30 | — | 00122001 |
| HT1080 MIC 1 | NC-MiC1 | 00122002 |
| HT1080-MIC 2 | NC-MiC2 | 00122003 |
| HT1080-MIC 3 | NC-MiC3 | 00122004 |
| HT1080-MIC 4 | NC-MiC4 | 00122005 |
| HT1080-MIC 5 | NC-MiC5 | 00122006 |
| HT1080-MIC 5a | NC-MiC5a | 00122007 |
| HT1080-MIC 5b | NC-MiC5b | 00122008 |

The present invention is further described by the following non-liming Examples.

### EXAMPLE 1

### Cell Culture and Chemicals

BE2Cl-18-5f (abbreviated 5f) was cultured as previously described (27). HT1080 and derivatives were cultured in DMEM (Gibco BRL) with 10% v/v fetal calf serum (FCS). Hygromycin (Roche), Puromycin (Sigma corp), or Zeocin (Invitrogen) were added to medium at concentrations of 250 μg/ml, 1 μg/ml, or 200 μg/ml, respectively. Medium was supplemented with 100 U/ml penicillin and 100 mg/ml streptomycin (Gibco BRL). All cell lines were maintained at subconfluence. Microtubule depolymerizing agents colcemid (Gibco BRL) or nocadazole (Sigma) were added to medium at concentrations of 10 μM or 0.1 μg/ml, respectively, for 1 or 6-12 hours, respectively, prior to cell harvesting. All chemicals used were of molecular biology grade and purchased from commercial sources.

### EXAMPLE 2

### Transfection of Cell Lines

Transfections of 5f and ZB30 cell lines were carried out using electroporation. Briefly, 10⁷ log-phase cells were harvested, washed twice in PBS and resuspended in 800 μl electroporation buffer (20 mM Hepes, pH 7.0, 150 mM NaCl, 5 mM KCl, 6 mM glucose, 2.5 mM NaOH, 1 mM Na₂HPO₄). 20 μg of linearized DNA was mixed with cells in a 0.4 cm cuvette and cells were electroporated at 1.2 kV, 25 uF using a Biorad Gene Pulser Electroporator. Transfection of HT1080 or derivative cells was carried out using either electroporation as previously described (30) or lipofection. For lipofection, the cells were plated at 1-3×10⁵ cells/ml in 40 ml on a 150 mm plate one day prior to transfection. This resulted in 50-70% confluency on the day of the experiment. 2 ml of serum-free DMEM was used to dilute 100 μl Fugene 6 transfection reagent (Boehringer Mannheim) and the suspension was incubated at 37° C. for 5 min. 20 μg of DNA was also diluted in 2 ml of serum-free DMEM. The diluted Fugene 6 was then added onto the DNA suspension drop-wise. The mixture was gently tapped and incubated for 15 min at room temperature. Following incubation, the DNA/Fugene 6 mixture was added onto the cells drop-wise. Cells were seeded into 96-well plates and selection was applied 24-48 hours post transfection.

### EXAMPLE 3

### Microcell Mediated Chromosome Transfer to HT1080

Microcell fusion was carried out as previously described (38). Log-phase donor ZB30 cells were incubated with colcemid (1 μg/ml) overnight. Cells were harvested after 48 hours and resuspended in percoll/serum-free DMEM (1:1) supplemented with 20 μg/ml of cytochalasin B (Sigma). The cell suspension was then subjected to centrifugation at 18,000 rpm for 90 min at 32° C. in oakridge tubes (Nalgene). Both bands of cell-mix were pelleted and washed once with serum-free DMEM prior to filtration through isopore membranes of 30, 8 and 5 μM (Millipore Corp., MA). The microcells were then resuspended in serum-free DMEM containing 10 μg/ml PHAP (Difco, West Molesey, UK), and were allowed to agglutinate with the recipient HT1080 cells for 45 min at 37° C. Following agglutination, cells were fused by addition of 50% w/v PEG (Boehringer Mannheim) and incubated for 2 min at room temperature followed by rinsing with serum-free DMEM. After incubation, cells were cultured overnight in DMEM containing 10% v/v FCS. The medium was then replaced with DMEM containing 200 [μg/ml of zeocin and cells were maintained in selection for a period of 14 days before colonies were picked for further characterization.

### EXAMPLE 4

### FISH/Immunofluorescence

Combined FISH/Immunofluorescence was carried out using a modified procedure previously described (25,51). FISH using pan-α-satellite probe pTRA7 and PNA-FISH of telomeric sequences (Perspectives Biosystem, MA) were carried out as previously described (39,40). Epifluorescence microscopy was performed on a Zeiss Axoplan II (Carl Zeiss, Carnegie, Australia) mounted with appropriate filter sets. Images were digitally acquired using a cooled charged-coupled device video camera (SenSys 2, Photometrics, Tuscon, Ariz., USA) connected to a PowerMac G3 personal computer controlled by the software IP Lab Version 2.5.5 (Scanalytics Inc., Fairfax, Va.,USA). Chromosome painting experiments were carried out using WCP Chromosome Paint Kit (Vysis Inc.) according to the manufacturer's instructions. Subchromosome-10 paints were derived from somatic cell radiation hybrid genomic DNA obtained from M. Rocchi (University of Bari). Inter-Alu amplification of somatic cell hybrid DNA was carried out using primers 5′ GGATTACAGGYRTGAGCCA [SEQ ID NO:1] and 5′ RCCAYTGCACTCCAGCCTG [SEQ ID NO:2] as previously described (41). Labelling of paint-probes and FISH were carried out using standard techniques.

Polyclonal anti-CENP-A, monoclonal anti-CENP-B, polyclonal anti-CENP-C, and CREST-6 antisera have been previously described (42,43,44,27). Polyclonal anti-CENP-E (45), anti-CENP-F (46), and anti-hBUB1 (47) were provided by T. J. Yen (Fox Chase Cancer Center), polyclonal anti-hZW10 (48) by B. Williams and M. L Goldberg (Cornell University), polyclonal p55CDC (49) by J. Weinstein (Amgen Corp) and polyclonal anti-TRF1 (32) by Titia de Lange (Rockefeller University, NY). Antisera CREST-6 was from a patient with the autoimmune CREST disease, containing antibodies against centromere components including CENP-A and CENP-B (51). Other secondary antibodies were purchased from the Jackson ImmunoResearch Laboratory Inc. (West Grove, Pa., USA).

### EXAMPLE 5

### Truncation Constructs

Truncation constructs contained either pGK:hygromycin, pGK:puromycin, or pGK:neomycin resistance gene cassettes. A 2 kb array of human telomeric repeats was obtained from pBS Sal-tel(5) plasmid (28,50). Genomic cosmid clones containing DNA corresponding to the p′ and q′ arms of mardel(10) have been previously described. 5-10 kb fragments lacking high-copy repeat DNA sequences (as evidenced by a lack of COT-1 hybridization following Southern hybridization), were subcloned in both orientations into truncation vectors using standard techniques. Alternatively genomic DNA was PCR amplified directly from BAC clones using Long Range PCR kit (Boehringer Mannheim) for cloning into truncation vectors. All truncation constructs were made in palter (Promega Corp.) vector backbone.

### EXAMPLE 6

### Concatamerization of Zeocin Resistance Marker

A zeocin resistance cassette from the pZeoSV2(+) plasmid (Invitrogen) was PCR amplified with the addition of flanking NotI restriction sites. This was cloned into pGEM-T (Promega Corp.). 100 μg of this plasmid was digested with NotI, and purified by phenol/chloroform extraction. To produce concatamers, 10 μl ligase buffer, 40 units of T4 ligase, and 5 μl of 100 mM ATP were added to the digested DNA in a total volume of 100 μl. The ligation reaction was carried out overnight and 1 μl of the completed reaction was tested by pulsed field gel electrophoresis to determine the size of the resulting concatamers. DNA was phenol/chloroform-extracted and ethanol-precipitated prior to transfection.

### EXAMPLE 7

### p′ Arm Truncation Screening Using Dot Blot Hybridization

Cells in 24-well plates were harvested by trypsinization and transferred directly to a Minifold dot blotting apparatus (96 per blot; Schleriche and Schuell Inc.) containing Hybond N+ (Amersham Corp.) under vacuum. Following transfer, Hybond N+ was denatured for 10 min (1M NaOH, 1M NaCl), neutralized twice for 5 min (1 M Tris-HCl, 1.5 M NaCl) followed by a final wash in 2×SSC. Membranes were then baked for 1.5 hours at 80° C. and were probed using ³²P-labelled pAlter vector (Promega Corp.) DNA using standard techniques.

### EXAMPLE 8

### Mardel(10) Tagging and Transfer Into HT1080 Cells

A CHO-based somatic cell hybrid line (designated 5f) containing the mardel(10) chromosome (27) was used. Initial telomere-associated chromosome truncation (TACT) using a system similar to that previously described (9,12,28) was performed on 5f employing the truncation vectors shown in FIG. 1A (described below). An exhaustive screening of over 25,000 drug-resistant colonies transfected with different truncation vectors yielded no positive truncation events, suggesting that the 5f cell line was not a suitable host for TACT. The inventors then decided to transfer the mardel(10) chromosome into human HT1080 cells since this cell line is known to be homologous recombination proficient (29,30), shows telomerase activity (31,32,33), and is a good recipient for microcell-mediated chromosome transfer (34,35). A random insertion approach was first used to tag mardel(10) in 5f cells with a zeocin resistance gene cloned in pGEM-T vector. In order to facilitate subsequent FISH screening for chromosome tagging, the zeo^{R}/pGEM-T construct containing zeocin resistance gene was concatamerized and only DNA fragments larger than 50 kb were used to transfect 5f cells. Screening 63 individual zeocin-resistant colonies identified a single cell line (designated ZB30) in which mardel(10) was tagged at its distal q′ region (FIG. 2A). This cell line was used as a donor in microcell-mediated chromosome transfer into HT1080 cells. 15 out of 60 zeocin-resistant microcell-fusion colonies were shown to contain mardel(10). Extensive FISH and immunofluorescence analyses of these cell lines using BACs from the neocentromere region, pan-α-satellite, CHO genomic DNA, and a host of anti-centromere antibodies confirmed that the mardel(10) chromosome and its neocentromere were intact, and that no integration of any α-satellite repeats or CHO DNA had occurred on the chromosome. One of these cell lines, designated ZBHT-14, was employed in subsequent truncation experiments.

In addition to cell lines such as ZBHT-14 which have acquired an intact mardel(10), a number of lines containing randomly truncated derivatives of this chromosome were also detected in the fusion products; the generation and transfer of such broken chromosomal fragments appeared to be a relatively common occurrence associated with the microcell fusion procedure (36). One cell line (designated NC-MiC1) was of interest because it carried a mini-chromosome that was shown by FISH to contain approximately 2 Mb of DNA around the previously mapped 10q25 neocentromere domain (27) (FIG. 1C). This cell line was retained for further characterization and truncation studies.

### EXAMPLE 9

### Truncation Constructs

A complete physical map was prepared containing over 50 BAC and cosmid clones covering approximately 3 Mb of the 10q25 neocentromere region (FIG. 1C). Based on this map, a number of truncation constructs containing different targeting DNA from this region were designed. These constructs contained either hygromycin (for q′-arm truncation), neomycin or puromycin (for p′-arm truncation) resistance genes, terminal cloned human telomere sequences, and 5-10 kb of targeting DNA from either the q′ or p′ arm of mardel(10) (FIG. 1A). Initial TACT experiments were performed on the q′ arm, followed by truncation of the p′ arm (FIG. 1B).

### EXAMPLE 10

### Truncation of q′ Arm

ZBHT-14 and NC-MiC1 cell lines were used in q′ truncation experiments. Both cell lines were transfected with a hyg^{R} (hygromycin resistance) truncation-construct containing a 6-kb targeting DNA derived from the Y3C94 cosmid (which overlaps with BAC B79E16; FIG. 1C). This targeting site is situated approximately 0.2 Mb from the anti-centromere antibody binding NC domain (27).

For the ZBHT-14 cell line, initial screening for q′ truncation of mardel(10) was performed by plating the resulting hygromycin-resistant colonies in zeocin. This allowed the identification of cell lines which have lost the zeocin-containing chromosome portion of mardel(10) and were, therefore, zeocin sensitive. From a total of 7,300 hygromycin-resistant colonies, 210 were shown to be zeocin sensitive. FISH analysis revealed that most of these colonies contained random truncations or other unknown rearrangements. One cell line (designated NC-MiC2) appeared to have undergone the desired targeted truncation and was characterized further.

FISH analysis of NC-MiC2 using a host of known cosmid or BAC clones mapped along the 10q25 neocentromere region revealed that clones on or proximal to the targeting DNA were present (e.g. Y3C94; FIG. 2B), whereas all clones distal to the targeting DNA were absent (e.g. Y3C109; FIG. 2C) (summarized in FIG. 1C). To more closely determine the site of truncation, three ˜10-kb PCR fragments (F1-F3) immediately adjacent to either side of the intended targeting site (FIG. 1D) were prepared and used in FISH analysis. The results indicated that only the fragment (F1) located proximally to the targeting DNA gave a positive signal (FIG. 2D), whilst the two distal fragments F2 and F3 were both negative (FIG. 2E). Further FISH using TTAGGG telomere-repeat peptide nucleic acid (PNA) probe demonstrated seeding of telomeric sequences on the truncated chromosome (FIG. 2H). Low-stringency FISH using pan-α-satellite probe and immunofluorescence using anti-CENP-B antibody (FIG. 2F) confirmed that no α-satellite DNA has been inserted into the truncated chromosome. These data strongly support a targeted-truncation event leading to the removal of most of the q′ arm of mardel(10) in the production of NC-MiC2.

Mitotic stability of NC-MiC2 was determined by comparing cells grown in the presence or absence of hygromycin over 20 cell divisions. A similar retention rate of 85% was observed for cells grown with or without drug selection, suggesting that NC-MiC2 was mitotically stable. To further investigate whether this chromosome maintained a functional neocentromere, immunofluorescence studies were carried out using CREST-6 autoimmune serum (27) and specific antibodies to the histone H3-like protein CENP-A, CENP-C, and the kinesin-like motor protein, CENP-E. Strong antisera signals that colocalized with the E8 BAC previously mapped to the neocentromere (NC) domain (37) were observed for all the proteins tested (e.g. FIG. 2G) thereby demonstrating neocentromere function on NC-MiC2.

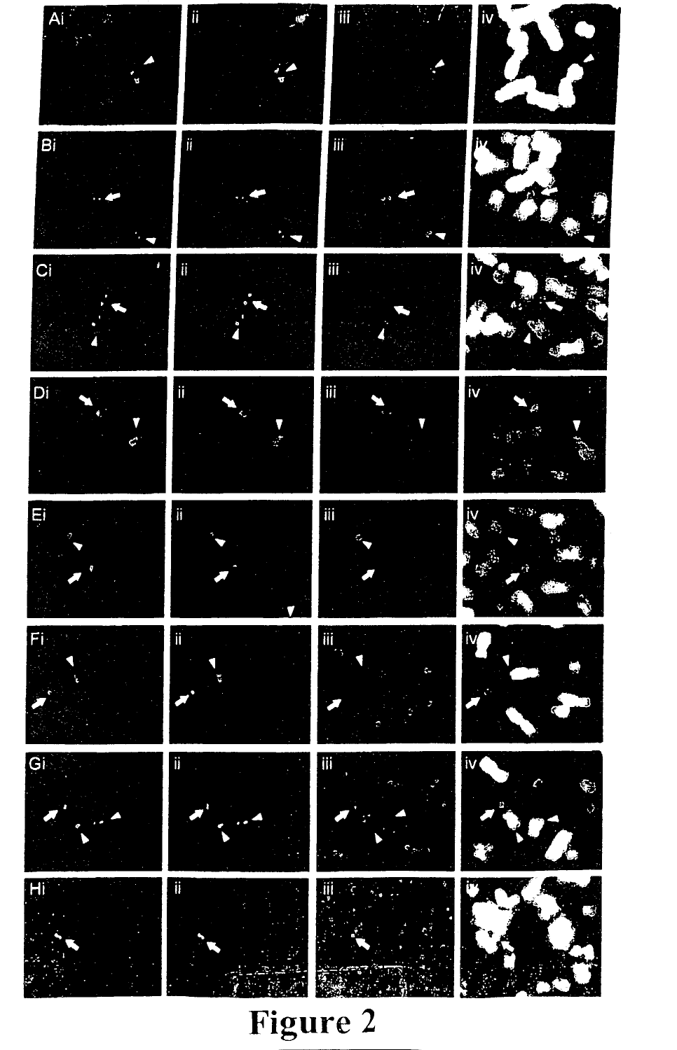

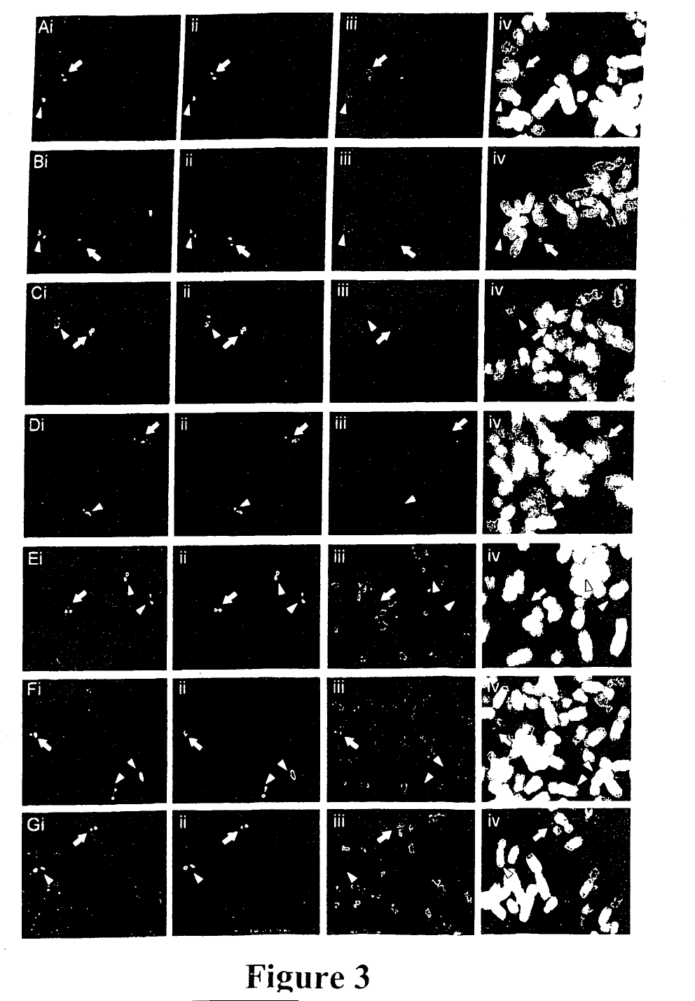

Transfection of the NC-MiC1 cell line with the Y3C94 DNA-containing q′ truncation construct resulted in over 1000 hygromycin-resistant colonies. These were screened for the loss of the vector DNA contained in the construct (see FIG. 1A and Example 7) that was indicative of a targeted truncation event. Detailed FISH analysis of one resulting cell line, NC-MiC3 with q′ cosmids and BACs, and the PCR probes F1-F3, demonstrated a correctly targeted truncation at the intended Y3C94 site (examples of FISH results are shown in FIGS. 3A-B, and summarized in FIG. 1C). Further p′ mapping and analysis of NC-MiC3 are described below.

### EXAMPLE 11

### Truncation of p′ Arm

NC-MiC2 was subjected to further truncation using constructs carrying puromycin or neomycin markers and targeting DNA from 3 different p′ regions (FIG. 1C). Puromycin- or neomycin-resistant colonies, generated through several independent transfection experiments, were screened for possible targeting by probing for loss of vector DNA as described above, as well as by dual-color FISH using E8 BAC and a distal p′-arm BAC (B10K1) (see FIG. 1B) to identify truncated NC-MiC2 derivatives. This analysis indicated that most colonies did not appear to contain the intended targeted chromosomal truncations of NC-MiC2. However, two cell lines (NC-MiC4 and NC-MiC5) were identified that showed truncation at p′ sites relatively close to the neocentromere antigen-binding region (27). These were characterized in greater detail.

### EXAMPLE 12

### Structure of NC-MiCs 3, 4 and 5

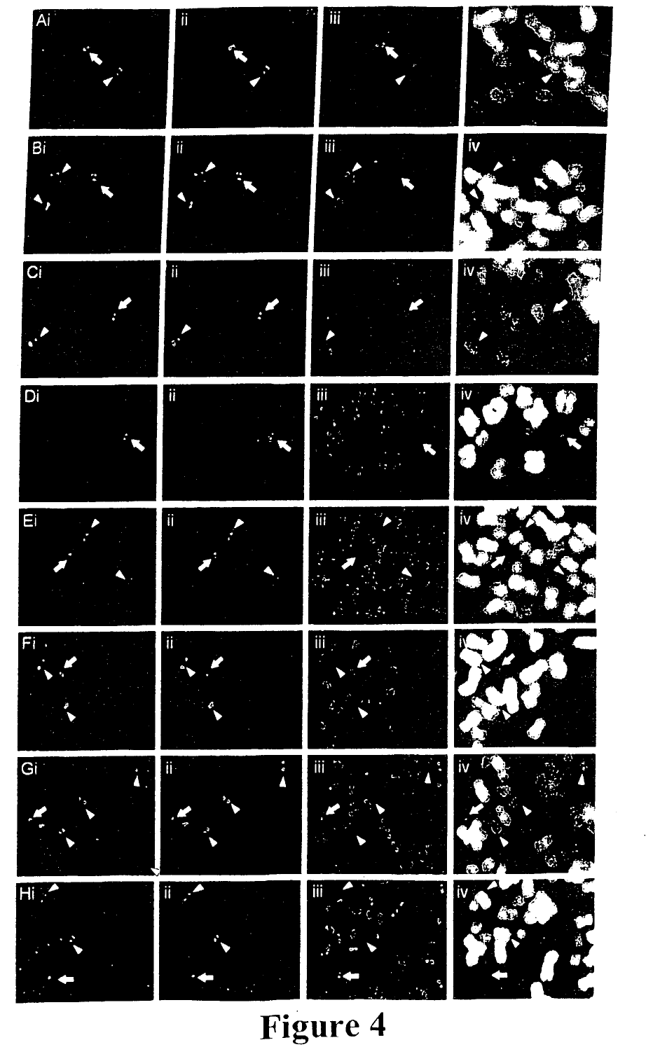

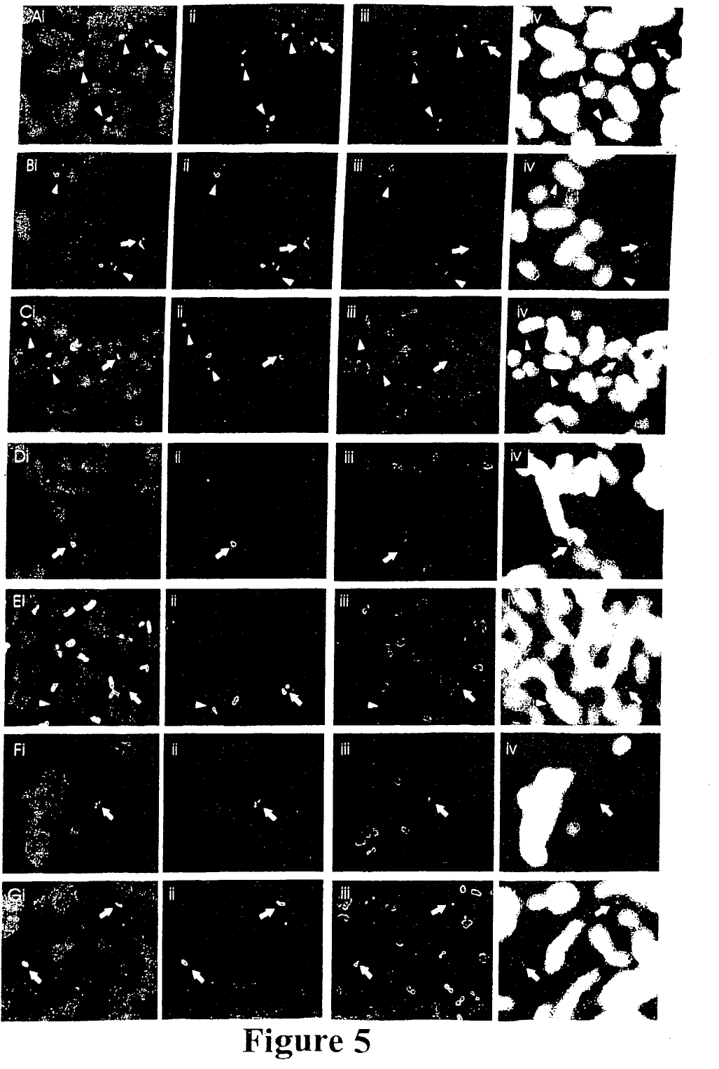

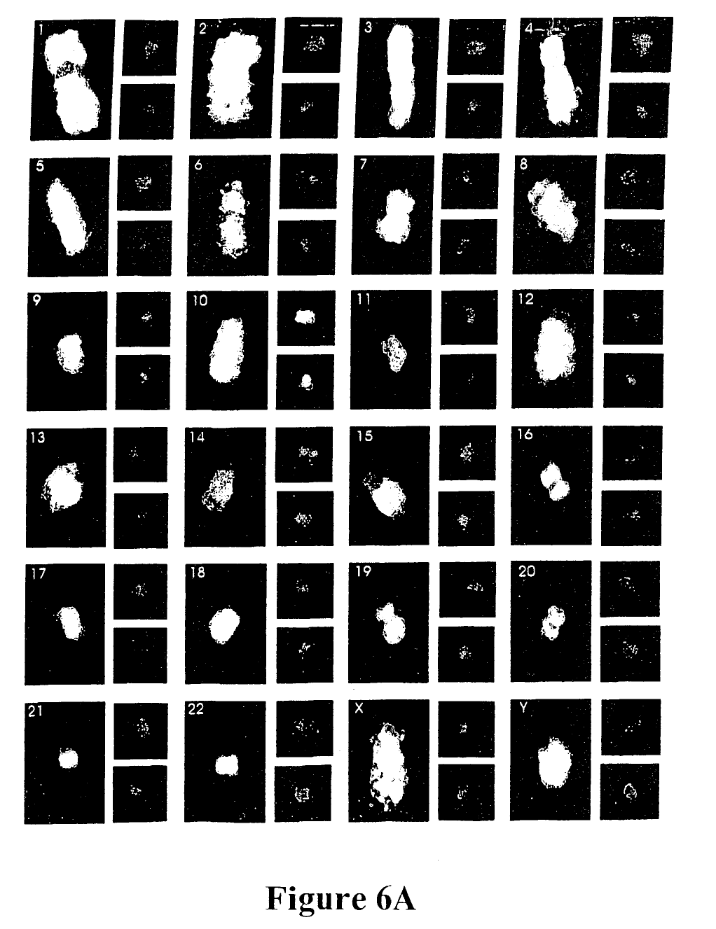

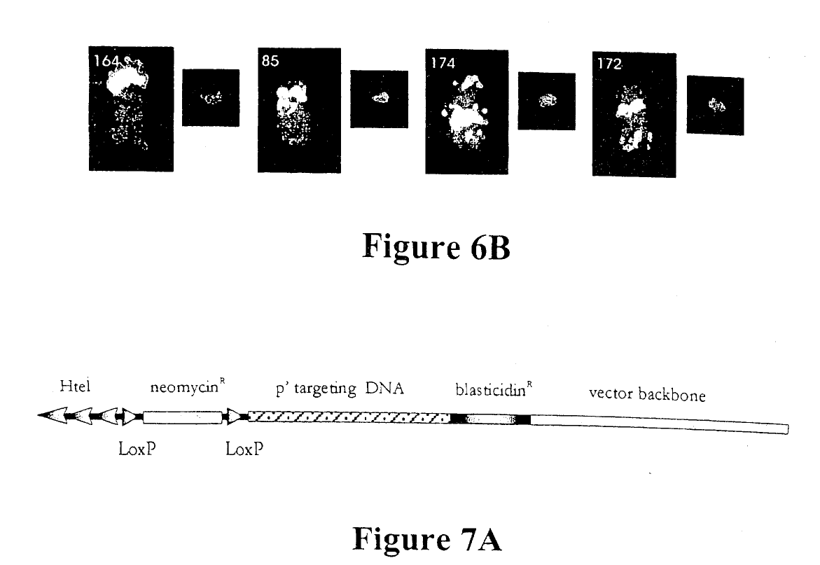

FIG. 1C summarizes the results for detailed FISH mapping of NC-MiCs 3, 4 and 5 using probes of known map position (some examples are shown in FIGS. 3-5). All three NC-MiCs showed the expected q′ truncation within Y3C94. On the p′ arm, chromosome truncation was seen between BACs Y13C12(present)/B179N3(absent), Y13C12 (present)/B43A11(absent), and BA48L24(present)/BA69K10(absent), for NC-MiC3 (and its predecessor NC-MiC1), NC-MiC4, and NC-MiC5, respectively (FIGS. 3C/D, 4A/B, and 5A/B). The intensity of the positive FISH signals on the different NC-MiCs were indistinguishable from those seen on the normal chromosomes 10 in HT1080 cells for all the cosmid and BAC probes tested, suggesting that no duplication of DNA has occurred during formation of the NC-MiCs. Low-stringency FISH hybridization using pan-α-satellite DNA probe (FIGS. 3E, 4C and 5C), and immunofluorescene using anti-CENP-B antibody (FIGS. 4E and 5D) demonstrated the absence of centromeric satellite DNA. When the different NC-MiC cell lines were analyzed by FISH using whole-chromosome paints for all 24 human chromosomes, only the chromosome 10-paint produced positive signals on the NC-MiCs, suggesting that no detectable amount of genomic DNA from other chromosomes have been translocated onto the NC-MiCs during their formation (FIG. 6A). Further analysis using a number of subchromosome 10-paints (see FIG. 1B for locations) similarly demonstrated the absence of DNA from non-10q25 regions on the NC-MiCs (FIG. 6B). It can, therefore, be concluded that NC-MiCs 3, 4 and 5 each contain single-copy DNA derived solely from the 10q25 neocentromere region, with total sizes estimated of approximately 1.6, 1.6 and 0.8 Mb, respectively (FIG. 1C).

High-molecular weight genomic DNA was prepared from the different cell lines and subjected to pulsed field gel electrophoresis under varying conditions that resolved DNA of up to 6 Mb. Only the NC-MiC3 migrated into the gel, suggesting that this is a linear mini-chromosome and raising the possibility that the other NC-MiCs are circular structures. Comparison with yeast chromosome markers indicated a size of 1.6 Mb on the PFGE gel for NC-MiC3. FISH using a pan-telomere probe or immunofluorescence using an antibody to the telomere repeat-binding factor TRF1 produced signals on telomeric ends of all normal chromosomes in HT1080 cells but not on any of the NC-MiCs, including the linear NC-MiC3 (e.g. FIG. 4D). This could be due to inability of the technique to detect low levels of telomere sequences and/or the circular nature of the NC-MiCs.

### EXAMPLE 13

### NC-MiC Stability and Neocentromere Activity

The mitotic stability of NC-MiCs 3, 4 and 5 was assayed following ≧20 cell divisions in culture media with and without selection. BAC E8 was used in FISH experiments to identify the NC-MiCs and 100 cells were scored for each cell line. For both NC-MiC3 and NC-MiC4, similar retention rates of approximately 80% were observed in the presence or absence of drug selection, suggesting that both chromosome derivatives were stable even in the absence of selection pressure. A retention rate of 36% was initially observed for the NC-MiC5 cell line with selection. Following removal of selection and culturing for 20 divisions, 37% of cells retained NC-MiC5, again suggesting mitotic stability in the absence of selective pressure; the reduced retention rate of NC-MiC5 compared to NC-MiCs 3 and 4 was most likely related to inherent genomic instability seen in this particular cell line.

As NC-MiC5 appeared to be the smallest derivative constructed, and because of the observed background genome instability in the original cell line, the inventors subcloned this line and examined the stability of NC-MiC5 in resulting clones. Two of the subclones (NC-MiC5a and NC-MiC5b) exhibited greatly increased stability of the NC-MiC5. Following ≧50 cell divisions in the absence of drug selection, these two cell lines demonstrated retention rates of 90% and 93%, respectively. These retention rates were not significantly different from those (90% and 91%, respectively) seen in cells passaged for the same number of divisions in the presence of selection. NC-MiC5a cells contained either one copy (76% of cells) or two copies (14% of cells) of the minichromosome, while only one copy was consistently detected in the NC-MiC5b cells. The structures of the NC-MiC5a and 5b were shown by detailed FISH analysis to be identical to the original NC-MiC5 chromosome (FIGS. 1C and 5).

Immunofluorescence detection was used to investigate the functional status of the neocentromeres on the NC-MiCs. Antisera to a host of centromere-associated proteins, including CENP-A, CENP-B, CENP-C, CENP-E, CENP-F, hZW10, p55CDC, and BUB1 were tested. All proteins with the exception of CENP-B were clearly detected on each of the NC-MiCs (some examples are shown in FIGS. 3F-G, 4F-H, 5E-G). These protein-distribution profiles were indistinguishable from those previously established in the parental mardel(10) chromosome (25,27), confirming that the NC-MiC derivatives contained full neocentromere activity.

### EXAMPLE 14

### Cell Culture and Transfection of NC-MiC2

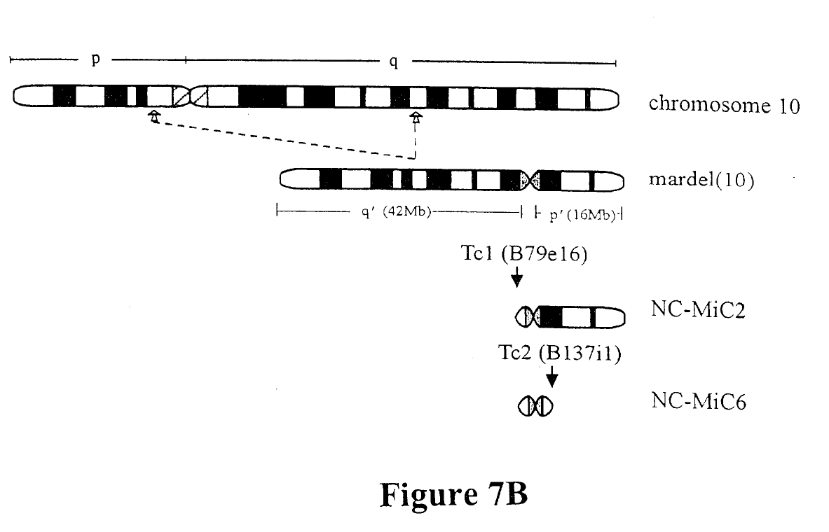

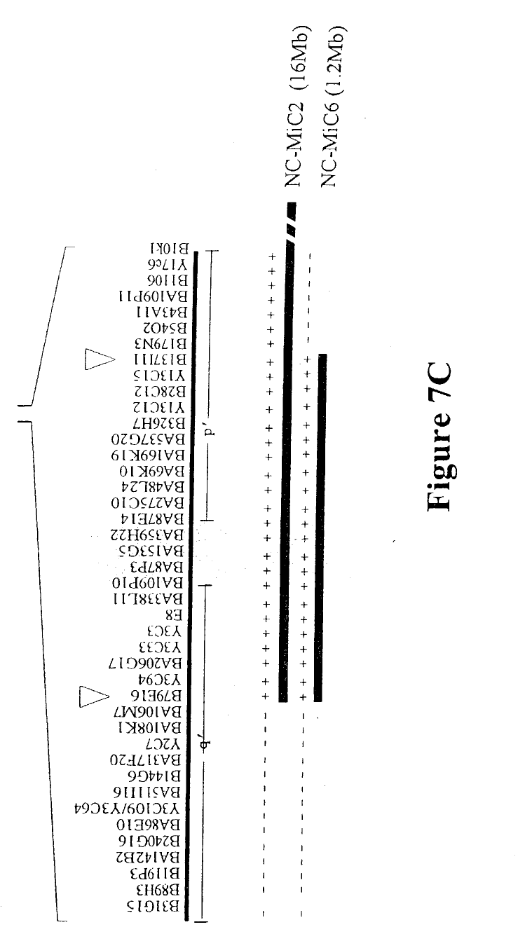

NC-MiC2 was cultured in DMEM (Gibco BRL) with 10% v/v FCS. Hygromycin (Roche) was added to medium at a concentration of 250 μg/ml. Transfection of NC-MiC2 was carried out using electroporation or lipofection. Electroporation was performed (0.4 kV, 250 uF) using a Biorad Gene Pulser Electroporator. For lipofection, the cells were plated one day prior to transfection to give 60-70% confluency at the time of transfection. Two ml of diluted Fugene 6 transfection reagent (100 μl in a total of 2 ml containing 20 μg DNA) (Boehringer Mannheim) was added onto cells drop-wise. The DNA used in transfection was a TACT construct containing human telomeric sequence htel, two loxP sites flanking a neomycin resistance gene, targeting genomic DNA and a blasticidin resistance gene (FIG. 7A). The antibiotic selection was applied 24-48 hours post-transfection at the concentration of 250 μg/ml for a period of 14 days before the colonies were picked for further characterization.

### EXAMPLE 15

### Truncation of p′ Arm of NC-MiC2

TACT experiments were performed on the p′ arm in NC-MiC2 cells using a neomycin-resistance truncation-construct containing a 4 kb targeting DNA derived from the Y13C15 cosmid/B137i1 BAC, two loxP sites, neomycin resistance gene and a blasticidin resistance gene outside the targeting DNA. A successful targeting event would result in the loss of blasticidin resistance gene. Of 10,000 neomycin-resistant cell lines, approximately 10% were blasticidin sensitive. Sensitivity to blasticidin was determined by culturing the clones in 5 μg/ml of blasticidin. Clones that were neomycin resistant but blasticidin sensitive were subjected to FISH analysis.

### EXAMPLE 16

### Characterization of NC-MiC6

One cell line (NC-MiC6) appeared to have undergone targeted truncation and was characterized extensively by FISH. Cosmids and BACs proximal to the targeting site were found to be present, whereas all clones distal to this site were absent (summarized in FIG. 7C). Based on the inventors' FISH and recently available genome sequence data, the inventors estimated the size of NC-MiC6 to be 1.2 Mb.

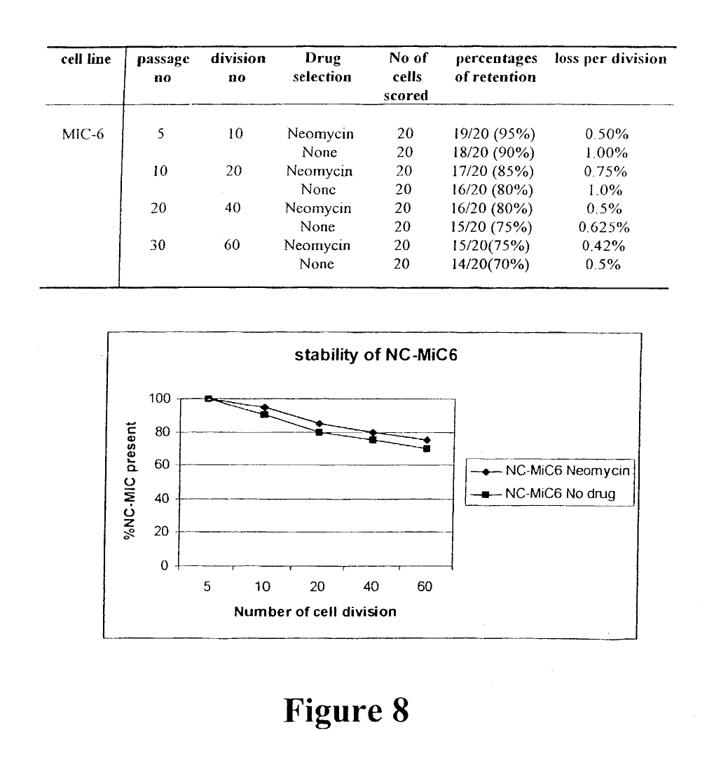

No α-satellite (pTRA7) and CENP-B binding were detected on the truncated chromosome. Immunofluorescence using CREST-6 autoimmune serum confirmed neocentromere activity on NC-MiC6. These data support, therefore, a targeted-truncation event that removed most of the p′ arm of mardel(10) in NC-MiC2. The mitotic stability of NC-MiC6 was assayed with and without selection for up to 60 divisions in culture. Retention rates of >80% were observed after 20 cell divisions, 75% at 40 cell divisions and with 70% ag 60 divisions (FIG. 8) in the absence of selection. In the presence of neomycin, 85% was maintained after 40 divisions and as high as 75% of NC-MiC6 was retained over 60 cell divisions, with loss rate of 0.42% per division, suggesting that NC-MiC6 was mitotically stable over time.

The presence of two loxP sites may be used for excision of neomycin resistance gene and insertion of new gene into specific site using cre-recombinase. The excision and insertion of genes is carried out either via transient transfection of plasmid containing the cre-recombinase gene or protein transfection of cre-recombinase.

### EXAMPLE 17

### Microcell Mediated Chromosome Transfer to Mouse Embryonic Stem ES Cells Expressing GFP (Green Fluorescent Protein) and to Mouse F9 Teratocarcinoma Cells

Mouse embryonic stem cells were cultured in ES medium (Gibco BRL) with 20% v/v FCS. Mouse F9 cells were cultured in DMEM (Trace Biosciences) with 10% v/v FCS. Transfection of ES cells was carried out using electroporation at 0.8 kV and 3 uF using a Biorad Gene Pulser Electroporator. The DNA used in transfection was pEGRP-N1 (Clontech) containing the gene encoding the green fluorescent protein and a neomycin resistance gene. Antibiotic selection was applied 24-48 hours post transfection (250 μg/ml G418 neomycin) for a period of 14 days before the colonies were picked and scaled up for screening.

Microcell fusion was carried out as previously described. Log-phase donor ZB30 cells arrested in colcemid for 48 hours were resuspended in percoll/serum-free DMEM (1:1) supplemented with 20 μg/ml of cytochalasin B (Sigma). The cell suspension was then subjected to centrifugation at 18,000 rpm for 90 min at 32° C. Both bands of cell-mix were pelleted, washed with serum-free DMEM and filtered through isopore membranes of 30, 8 and 5 μM (Millipore Corp., MA). Microcells were then fused with recipient neomycin-resistant ES-GFP or F9 cells by addition of 50% w/v PEG (Roche) for 2 min at room temperature. After incubation, cells were rinsed and cultured overnight in ES media containing 20% v/v FCS or DMEM containing 10% v/v FCS followed by addition of antibiotic selection (250 μg/ml G418 neomycin and 100 μg/ml of zeocin for ES cells and 100 μg/ml zeocin for F9 cells) 24 hrs later.

### EXAMPLE 18

### Characterization of ES-GFPmar (10)#1 and F9-4-5mar(10) Cell Lines

Extensive FISH analysis of ES-GFPmar(10)#1 and F9-4-5mar(1) demonstrated that the mardel(10) chromosome and the neocentromere contained therein were intact. No mouse centromeric/pericentromeric major and minor satellite DNA or genomic DNA was detected on the marker chromosome in either cell line. In addition, mardel(10) was the only human chromosome present in these cell lines. The stability of mardel(10) was assayed with selection for up to 45 divisions in culture and the marker chromosome showed mitotic stability in both cell lines.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

Various publications are cited herein, the contents of which are hereby incorporated by reference in their entireties.

### BIBLIOGRAPHY

1. Dobie et al. (1999) Curr. Opin. Genet. Dev. 9: 206-17.

2. Pidoux and Allshire, R. C. (2000) Curr. Opin. Cell Biol. 12: 308-19.

3. Tyler-Smith and Floridia (2000) Cell 102: 5-8.

4. Harrington et al. (1997) Nat. Genet. 15: 345-355.

5. Ikeno et al. (1998) Nat. Biotechnol. 16: 431-439.

6. Henning et al. (1999) Proc. Natl. Acad. Sci. U S A. 96: 592-597.

7. Ebersole et al. (2000) Hum. Mol. Genet. 9: 1623-1631.

8. Masumoto et al. (1998) Chromosoma 107: 406-416.

9. Farr et al. (1992) Nat. Genet. 2: 275-282.

10. Farr et al. (1995) EMBO J. 14: 5444-5454.

11. Mills et al. (1999) Hum. Mol. Genet. 8: 751-761.

12. Heller et al. (1996) Proc. Natl. Acad. Sci. U S A. 93: 7125-7130.

13. Yang et al. (2000) Hum. Mol. Genet. 9: 1891-902.

14. Shenetal. (1997) Hum. Mol. Genet. 6: 1375-1382.

15. Loupart et al. (1998) Chromosoma 107: 255-259.

16. Kereso et al. (1996) Chromosome Res. 4:226-39.

17. Csonka et al. (2000) J. Cell. Sci. 113: 3207-3216.

18. Telenius et al. (1999) Chromosome Res. 7: 3-7.

19. DeJong et al. (1999) Cytometry 35: 129-133.

20. Co et al.(2000) Chromosome Res. 8: 183-91.

21. Choo (1997) Am. J. Hum. Genet. 61: 1225-33.

22. Williams (1998) Nat. Genet. 18: 30-7.

23. Barry et al. (2000) Genome Res. 10: 832-838.

24. Choo (2000) Trends Cell Biol. 10: 182-188.

25. Saffery et al. (2000) Hum. Mol. Genet. 9: 175-185.

26. Warburton et al. (2000) Am. J. Hum. Genet. 66: 1794-1806.

27. du Sart et al. (1997) Nat. Genet. 16: 144-153.

28. Farr et al. (1991) Proc. Natl. Acad. Sci. U S A. 88: 7006-7010.

29. Porter and Itzhaki (1993) Eur. J. Biochem. 218: 273-81.

30. Itzhaki and Porter (1991) Nucl. Acids Res. 19: 3835-42.

31. Parris et al. (1999) Br. J. Cancer 79: 47-53.

32. van Steensel and de Lange (1997) Nature 385: 740-743.

33. Holt et al. (1996) Mol. Cell. Biol. 16: 2932-2939.

34. Klein and Bouck (1994) Cancer Genet. Cytogenet. 73: 109-12

35. Kugoh et al. (1990) Oncogene 5: 1637-1644.

36. Henning and Friedberg (1993) Methods in Molecular Genetics Vol 1. Acad. Press. London

37. Cancilla et al. (1998) Genomics 47: 399-404.

38. Killary and Lott (1996) Methods: A Companion to Methods in Enzymology 9: 3-11.

39. Lo and Choo (1999) Biotechniques 26: 408-412.

40. Lansdorp et al. (1996) Hum. Mol. Genet. 5: 685-691.

41. Liu et al. (1993) Cancer Genet. Cytogenet. 65: 93-99.

42. Saffery et al. (1999) Chromosome Res. 7: 261-265.

43. Hudson et al. (1998) J. Cell Biol. 141: 309-319.

44. Kalitsis et al. (1998) Proc. Natl. Acad. Sci. USA. 95: 1136-1141.

45. Thrower et al. (1995) EMBO J. 14: 918-926.

46. Liao et al. (1995) J. Cell Biol. 130: 507-518.

47. Jablonski et al. (1998) Chromosoma 107: 386-396.

48. Starr et al. (1997) J. Cell Biol. 138: 1289-1301.

49. Weinstein et al. (1994) Mol. Cell. Biol. 14: 3350-3363.

50. Cross et al. (1990) Nucl. Acids Res. 8: 6649-6657.

51. Voullaire et al. (1999) Am. J. Med. Genet. 85: 403-408.

52. Lo et al. (2001) EMBO J. 20: 2087-2096.

1. An isolated nucleic acid molecule comprising a sequence of nucleotides derived from a eukaryotic chromosome and encompassing a neocentromere, which nucleic acid molecule exists in a truncated form in a compatible cell or, when introduced by transfection as a pre-fabricated DNA entity into a compatible cell, is capable of replicating, acting as an extra chromosomal element, and segregating with cell division.

2. The isolated nucleic acid molecule of claim 1 wherein the eukaryotic chromosome is derived from a mammal, including a human or primate, a plant, an avian species, an insect, a worm, a fungus, a yeast or a reptile.

3. The isolated nucleic acid molecule of claim 2 wherein the eukaryotic chromosome is derived from a human.

4. The isolated nucleic acid molecule of claim 2 wherein the eukaryotic chromosome is derived from a livestock animal.

5. The isolated nucleic acid of claim 1 comprising a q′ arm of the mardel(10) chromosome or its equivalent truncated by targeted telomere-associated truncation and further comprising the p′ arm of the mardel(10) chromosome or its equivalent truncated by random truncation.

6. The isolated nucleic acid molecule of claim 5 wherein said nucleic acid molecule comprises a neocentromere at a location equivalent to between q24 and q26 on chromosome 10 or its equivalent.

7. The isolated nucleic acid molecule of claim 6 wherein said nucleic acid molecule comprises a neocentromere at a location equivalent to at or about q25 on chromosome 10 or its equivalent.

8. The isolated nucleic acid molecule of claim 7 wherein said nucleic acid molecule comprises a neocentromere at a location equivalent to at or about q25.2 on chromosome 10 or its equivalent.

9. The nucleic acid molecule of any one of claims 1, 6, 7 or 8 wherein the region corresponding to a neocentromere is substantially devoid of α-satellite DNA.

10. The nucleic acid molecule of claim 9 wherein the nucleic acid molecule is from about 0.5 to about 2.0 Mb in size.

11. The nucleic acid molecule of claim 10 wherein the nucleic acid molecule is from about 0.8 to about 1.6 Mb in size.

12. A method for identifying a neocentromere or a functional homolog thereof, said method comprising isolating DNA by chromatin immunoprecipitation with an antibody specific to mammalian CENP-A and/or CENP-C or an antibody capable of cross interacting with mammalian CENP-A and/or CENP-C, amplifying the DNA isolated by immunoprecipitation, incorporating a label into the amplified DNA, probing a DNA array comprising genomic DNA or its equivalent with the labeled, amplified DNA, and identifying and isolating clones which hybridize to said immunoprecipitated DNA.

13. The method of claim 12 wherein the mammal is a human, livestock animal, companion animal or laboratory test animal.

14. The method of claim 13 wherein the mammal is a human.

15. The method of claim 14 wherein the neocentromere is at a location equivalent to between p24 and p26 on chromosome 10 or its equivalent.

16. The method of claim 15 wherein the neocentromere is at a location at or about q25 on chromosome 10 or its equivalent.

17. The method of claim 16 wherein the neocentromere is at a location at or about q25.2 on chromosome 10 or its equivalent.

18. An isolated human neocentromere-based mini-chromosome (NC-MiC), said NC-MiC comprising a neocentromere or a latent, synthetic or hybrid form thereof which enables stable segregation during cell division.

19. The isolated mini-chromosome of claim 18 comprising a q′ arm of the mardel(10) chromosome or its equivalent truncated by targeted telomere-associated truncation and further comprising the p′ arm of the mardel(10) chromosome or its equivalent truncated by random truncation.

20. The isolated mini-chromosome of claim 19 wherein the neocentromere is at a location equivalent to between q24 and q26 on chromosome 10 or its equivalent.

21. The isolated mini-chromosome of claim 20 wherein the neocentromere is at a location at or about q25 on chromosome 10 or its equivalent.

22. The isolated mini-chromosome of claim 20 wherein the neocentromere is at a location at or about q25.2 on chromosome 10 or its equivalent.

23. A method for generating a mini-chromosome, said method comprising:
introducing into a human or mammalian cell which carries a chromosome containing a neocentromere, a truncation construct comprising a vector having telomeric sequences, a selectable marker, homologous targeting DNA on one or other of the q′ or p′ arm of the target chromosome;
selecting for cells expressing the selectable marker;
introducing into said cells a second truncation construct comprising the other of said q′ or p′ targeting DNA;
selecting for cells expressing the selectable marker associated with said second truncation construct; and
then isolating the truncated chromosome which comprises a neocentromere.

24. The method of claim 23 wherein the cell is a human cell.

25. The method of claim 24 wherein the neocentromere is at a location equivalent to between q24 and q26 on chromosome 10 or its equivalent.

26. The method of claim 24 wherein the neocentromere is at a location at or about q25 on chromosome 10 or its equivalent.

27. The method of claim 24 wherein the neocentromere is at a location at or about q25.2 on chromosome 10 or its equivalent.

28. An isolated cell comprising a chromosome having q′ and p′ arms flanking a neocentromere wherein one or both q′ and/or p′ arms are truncated.

29. An isolated cell line deposited at ECAAC under Accession 00122001 (CHO/BE ZB30).

30. An isolated cell line deposited at ECAAC under Accession 00122002 (HT1080-MIC 1).

31. An isolated cell line deposited at ECAAC under Accession 00122003 (HT1080-MIC 2).

32. An isolated cell line deposited at ECAAC under Accession 00122004 (HT1080-MIC 3).

33. An isolated cell line deposited at ECAAC under Accession 00122005 (HT1080-MIC 4).

34. An isolated cell line deposited at ECAAC under Accession 00122006 (HT1080-MIC 5).

35. An isolated cell line deposited at ECAAC under Accession 00122007 (HT1080-MIC 5a).

36. An isolated cell line deposited at ECAAC under Accession 00122008 (HT1080-MIC 5b).

37. The mini-chromosome generated by the method of claim 23, wherein the mini-chromosome is an NC-MiC.

38. The mini-chromosome generated by the method of claim 23, wherein the mini-chromosome is a YAC.

39. The mini-chromosome generated by the method of claim 23, wherein the mini-chromosome is a HAC.

40. The mini-chromosome generated by the method of claim 23, wherein the mini-chromosome is a MAC.

41. The mini-chromosome generated by the method of claim 23, wherein the mini-chromosome is a PLAC.

42. A method of gene transfer comprising introducing into a target cell the mini-chromosome of any one of claims 37, 38, 39, 40 or 41.
